(19) United States
(12) Patent Application Publication
(10) Pub. No.: US 2024/0115466 A1
(43) Pub. Date: Apr. 11, 2024

(54) PROCESS FOR PREPARING A SHAMPOO FROM AN ANHYDROUS SOLID SURFACTANT COMPOSITION

(71) Applicant: L'OREAL, Paris (FR)

(72) Inventors: Frederik PINAY, Saint Ouen (FR); Mélanie OTT, Saint Ouen (FR); Maxime ROYER, Saint Ouen (FR); Damien DRILLON, Saint Ouen (FR)

(73) Assignee: L'OREAL, Paris (FR)

(21) Appl. No.: 18/255,789

(22) PCT Filed: Dec. 3, 2021
(86) PCT No.: PCT/EP2021/084236

§ 371 Date: Jun. 2, 2023

(30) Foreign Application Priority Data
Dec. 3, 2020 (FR) 2012602

(51) Int. Cl. A61K 8/02; A61Q 5/02

(52) U.S. Cl. CPC A61K 8/022; A61Q 5/02; A61K 2800/31; A61K 2800/596; A61K 2800/882

(57) ABSTRACT

The present invention relates to a process for preparing a composition for washing keratin materials, in particular human keratin fibres such as the hair, comprising the mixing of an anhydrous solid composition comprising one or more anionic surfactants and one or more amphoteric or zwitterionic surfactants with water, followed by agitation and then leaving to stand the aqueous composition obtained from this mixing.

The present invention relates to a process for preparing a composition for washing keratin materials, in particular human keratin fibres such as the hair, comprising the mixing of an anhydrous solid composition comprising one or more anionic surfactants and one or more amphoteric or zwitterionic surfactants with water, followed by agitation and leaving to stand the aqueous composition obtained from this mixing.

In the field of hair hygiene, products for washing and/or caring for keratin fibres are predominantly, if not to say all, packaged in plastic packagings, for example in non-biodegradable plastic bottle or plastic bag form.

At the present time, the cosmetics field is dependent on these plastic packagings which are sparingly recycled or difficult to recycle, and the solutions for dispensing with these packagings are rare.

Packagings based on paper or biodegradable materials for aqueous compositions for washing and/or caring for keratin fibres are not entirely satisfactory. The reason for this is that this type of packaging may disintegrate in the presence of water, which does not enable these compositions to be stored or enable good stability over time to be conserved.

However, out of concern for preserving the environment, consumers are increasingly vigilant regarding the plastic footprint of their purchases and are in search of cosmetic products packaged without plastic materials or petrochemical-based materials, and in smaller packagings.

Moreover, still out of concern for preserving the environment, consumers are also mindful of the carbon footprint of their purchases, i.e. the amount of carbon dioxide generated from the production of the cosmetic product up to its use, passing through the distribution of the product.

In the past, solid compositions in the form of cakes or bars were able to be marketed. However, these compositions in cake or bar form were not entirely satisfactory, notably as regards the application of the solid composition to the hair or the body. The reason for this is that it was seen that it is less easy for the user to apply uniformly to the hair/body an effective dose of a solid composition in cake or bar form, than the same dose with a conventional aqueous composition.

There is thus a real need to develop processes for preparing cosmetic products with a plastic footprint and a carbon footprint that are smaller than those of the conventional preparation processes. In particular, there is a real need to develop processes for preparing washing compositions that are capable of reducing the generation of carbon dioxide throughout the life of the product, and also to obtain washing compositions which give keratin materials good working performance qualities, notably in terms of washing power, suppleness, feel, softness, sheen and disentangling of keratin fibres.

In addition, it is also advantageous for these preparation processes to be able to recreate aqueous washing/conditioning compositions that are ready-to-use for keratin materials, and for these ready-to-use aqueous compositions to be able to be recreated easily by the user himself, and to do so well in advance of their use.

These objectives are achieved by the present invention, one subject of which is notably a process for preparing a composition for washing keratin materials, comprising:
(i) at least one step of mixing:
   a. an anhydrous solid composition (A) comprising:
      one or more anionic surfactants, and
      one or more amphoteric or zwitterionic surfactants,
   b. with a composition (B) comprising water,
the weight ratio of the total content of composition (A) to the total content of composition (B) ranging from 0.01 to 0.5; and then
(ii) at least one step of agitating the aqueous composition (C) resulting from said mixing; and then
(iii) at least one step consisting in leaving the aqueous composition (C) to stand for at least 20 minutes before using it on the keratin materials.

The process according to the invention enables the user to prepare at home a ready-to-use aqueous composition for washing keratin materials, for example a shampoo, by mixing the anhydrous solid composition (A) with an aqueous composition (B) in a bottle, for example a glass bottle. The ready-for-use aqueous composition thus prepared can be used for several days and even several weeks after it has been prepared.

The anhydrous solid composition (A) has the advantage of being able to be packaged in non-plastic packagings, for example in bags made of paper or of biodegradable materials or metallic bags, and also of being packaged in smaller packagings than those of a conventional shampoo.

The anhydrous solid composition (A) also has the advantage of having a reduced weight and volume, relative to a conventional aqueous washing composition, for example a 250 mL shampoo packaged in a polyethylene terephthalate bottle. In other words, for a unit of anhydrous solid composition, equivalent to a unit of aqueous washing composition once reformulated with water or to 250 mL of conventional shampoo, the weight and volume of the anhydrous solid composition are lower.

These weight and volume reductions per unit of anhydrous solid composition (A) make it possible to significantly reduce the amount of carbon dioxide generated during the transportation of the compositions (A) from the production factory to the distribution points, or even to the homes of the consumers.

The reason for this is that reducing the volume per unit of composition (A) makes it possible to deliver more via the same transportation means (for example a truck or an aeroplane) and thus to reduce the amount of carbon dioxide generated, relative to each unit of composition (A).

Similarly, reducing the weight per unit of composition (A) makes it possible to reduce the total weight of the delivery for the same transportation means (for example a truck or an aeroplane) and thus the amount of combustible energies required for the delivery (for example gasoline or kerosene) and thus to reduce the amount of carbon dioxide generated, relative to each unit of composition (A).

It has also been found that the ready-to-use aqueous washing compositions thus obtained have good washing power and afford suppleness, a good feel, softness, sheen and ease of disentangling to keratin fibres.

Advantageously, the ready-to-use aqueous washing compositions thus obtained are transparent.

A subject of the invention is also a process for treating keratin materials, comprising the application to said keratin materials of the composition obtained via the preparation process according to the invention.

Other subjects, characteristics, aspects and advantages of the invention will emerge even more clearly on reading the description and the example that follows.

In the present description, and unless otherwise indicated:
the term “at least one” is equivalent to the term “one or more” and can be replaced therewith;
the term “between” is equivalent to the term “ranging from” and can be replaced therewith, and implies that the limits are included;
for the purposes of the present invention, the term “less than” and, respectively, the term “greater than” refer to an open range which is strictly less, or, respectively, strictly greater, and thus that the limits are not included.
according to the present patent application, the term “keratin materials” more particularly denotes keratin fibres, more preferentially human keratin fibres and even more preferentially the hair.

The Anhydrous Solid Composition (A)

The preparation process according to the invention comprises at least one step of mixing an anhydrous solid composition (A) with an aqueous composition (B).

Composition (A) is solid at room temperature (25° C.) and at atmospheric pressure (1.013×10⁵ Pa).

Composition (A) is anhydrous.

The term “anhydrous composition” means a composition comprising an amount of water of less than 5% by weight, preferably less than 3% by weight relative to the weight of the composition. Preferably, this water content is less than 1% by weight, better still less than 0.5% or even less than 0.3% by weight, relative to the weight of the composition. More particularly, the composition does not comprise any water (0%).

In particular, the anhydrous solid composition (A) does not comprise any water added during its preparation, the residual water that may be present possibly originating from the starting materials used during the preparation.

The anhydrous solid composition (A) may be in powder, paste, particle (for example spherical particles such as small beads or granules), compressed tablet, stick or cake form. Preferably, composition (A) is in powder or particle form, and more preferentially in powder form.

The term “powder” means a composition in pulverulent form, which is preferably essentially free of dust (or fine particles). In other words, the particle size distribution of the particles is such that the weight content of particles which have a size of less than or equal to 50 micrometres (content of fines), preferably less than or equal to 45 micrometres (content of fines) is advantageously less than or equal to 5% by weight, preferably less than 3% by weight and more particularly less than 1% by weight, relative to the total weight of the particles (particle size evaluated using a Retsch AS 200 Digit particle size analyser; oscillation height: 1.25 mm/screening time: 5 minutes). Advantageously, the size of the powder particles is between 5 μm and 3 mm, preferably between 10 μm and 2 mm, more preferentially between 50 μm and 1 mm and better still between 60 μm and 600 μm.

The term “paste” means a composition having a viscosity of greater than 5 poises (500 mPa·s) and preferably greater than 10 poises (1000 mPa·s), measured at 25° C. and at a shear rate of 1 s⁻¹; this viscosity possibly being determined using a cone-plate rheometer.

The term “particles” means small fractionated objects formed from solid particles that are aggregated together, of variable shapes and sizes. They may be in regular or irregular form. They may in particular be in spherical form (such as granules, granulates or beads) or in square, rectangular or elongated form such as sticks. Spherical particles are most particularly preferred. The size of the particles may advantageously be, in the largest dimension thereof, between 5 μm and 5 mm, preferably between 10 μm and 2 mm, more preferentially between 50 μm and 1000 μm and better still between 60 μm and 600 μm.

When the anhydrous solid composition (A) is not in powder or particle form, it preferably has a penetration force at 25° C. and 1 atm (1.013×10⁵ Pa) of greater than or equal to 200 g, notably greater than or equal to 300 g, or even 400 g and better still 500 g. The penetration force is determined by penetrometry. The texture analysis measurements are performed at 25° C. using a Stable Micro Systems TA.XT Plus texturometer. The penetrometry experiments are performed with a metal rod equipped with a screwed end piece, said end piece being a P/2N needle of 2 mm for the top part, connected to the measuring head. The piston penetrates into the sample at a constant speed of 1 mm/s, to a depth of 5 mm. The force exerted on the piston is recorded and the mean value of the force is calculated.

The anhydrous solid composition (A) may be in the form of a compressed anhydrous solid composition, notably compressed using a manual or mechanical press.

The density of the anhydrous solid composition (A) is preferably between 0.1 and 1, more preferentially between 0.2 and 0.8 and better still between 0.3 and 0.6.

By way of example, the density may be measured according to the following method. A given amount (mass, m) of powder is placed in a 250 mL measuring cylinder. The powder contained in the measuring cylinder then undergoes 2500 compressions and the volume (v) occupied by the powder is obtained. The volume (v) thus obtained is read on the measuring cylinder and the density (d) is then determined according to the formula d=m/v.

Anionic Surfactants

The anhydrous solid composition (A) comprises one or more anionic surfactants.

The term “anionic surfactant” means a surfactant which includes, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

The anionic surfactants may be chosen from sulfate, sulfonate and carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The carboxylate anionic surfactants that may be used thus include at least one carboxylic or carboxylate function (—COOH or —COO⁻).

The carboxylate anionic surfactants may be chosen from the following compounds: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof,

the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Preferentially, the carboxylate anionic surfactants are chosen, alone or as a mixture, from:
acylglutamates, notably of C₆-C₂₄ or even C₁₂-C₂₀, such as stearoylglutamates, and in particular disodium stearoylglutamate;
acylsarcosinates, notably of C₆-C₂₄ or even C₁₂-C₂₀, such as palmitoylsarcosinates, and in particular sodium palmitoylsarcosinate;
acyllactylates, notably of C₁₂-C₂₈ or even C₁₄-C₂₄, such as behenoyllactylates, and in particular sodium behenoyllactylate;
C₆-C₂₄ and notably C₁₂-C₂₀ acylglycinates;
(C₆-C₂₄)alkyl ether carboxylates, and notably (C₁₂-C₂₀)alkyl ether carboxylates;
polyoxyalkylenated (C₆-C₂₄)alkyl(amido) ether carboxylic acids, in particular those including from 2 to 50 ethylene oxide groups;

in particular in the form of alkali metal or alkaline-earth metal, ammonium or amino alcohol salts.

Among the above carboxylic surfactants, mention may be made most particularly of surfactants of sarcosinate type, notably chosen from (C₆-C₃₀)acyl sarcosinates of formula (I) below:

R—C(O)—N(CH₃)—CH₂—C(O)—OX (I)

with
X denoting a hydrogen atom, an ammonium ion, an ion derived from an alkali metal or an alkaline-earth metal or an ion derived from an organic amine, preferably a hydrogen atom, and
R denoting a linear or branched alkyl group of 5 to 29 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (I) that may be used in the present composition, mention may be made of palmitoyl sarcosinates, stearoyl sarcosinates, myristoyl sarcosinates, lauroyl sarcosinates and cocoyl sarcosinates, in acid form or in salified form.

The anionic surfactant(s) of sarcosinate type are advantageously chosen from sodium lauroyl sarcosinate, stearoylsarcosine, myristoylsarcosine, and mixtures thereof, preferably from stearoylsarcosine, myristoylsarcosine, and mixtures thereof.

Among the above carboxylic surfactants, mention may also be made of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (II):

R1-(OC₂H₄)ₙ—OCH₂COOA (II)

in which:
R1 represents a linear or branched C₆-C₂₄ alkyl or alkenyl radical, a (C₈-C₉)alkylphenyl radical, a radical R₂CONH—CH₂—CH₂— with R2 denoting a linear or branched C₉-C₂₁ alkyl or alkenyl radical;

preferably, R1 is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (II), in particular mixtures of compounds bearing different groups R1.

The polyoxyalkylenated alkyl(amido) ether carboxylic acids that are particularly preferred are those of formula (II) in which:
R1 denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (II) in which R1 denotes a C₁₂ alkyl radical, A denotes a hydrogen or sodium atom and n ranges from 2 to 10.

The sulfonate anionic surfactants that may be used include at least one sulfonate function (—SO₃H or —SO₃⁻).

The sulfonate anionic surfactants may be chosen from the following compounds: alkylsulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
C₆-C₂₄ and notably C₁₂-C₂₀ N-acyltaurates;
(C₆-C₂₄)acylisethionates, preferably (C₁₂-C₁₈)acylisethionates;

in particular in the form of alkali metal or alkaline-earth metal, ammonium or amino alcohol salts.

Preferably, the anionic surfactant(s) of sulfonate type are chosen from C₆-C₂₄ and notably C₁₂-C₂₀ N-acyltaurates, and in particular N-acyl N-methyltaurates, C₆-C₂₄ and notably C₁₂-C₁₈ acylisethionates, and also salts thereof and mixtures thereof.

More preferentially, the anionic surfactant(s) of sulfonate type are chosen from C₆-C₂₄ and notably C₁₂-C₁₈ acylisethionates, and also salts thereof and mixtures thereof.

The sulfate anionic surfactants that may be used include at least one sulfate function (—OSO₃H or —OSO₃).

The sulfate anionic surfactants be chosen from the following compounds: alkyl sulfates, alkyl ether sulfates, alkylamido ether sulfates, alkylaryl polyether sulfates, monoglyceride sulfates; and the salts of these compounds;

the alkyl groups of these compounds including from 6 to 30 carbon atoms, notably from 8 to 28, even better still from 10 to 24 or even from 12 to 22 carbon atoms; the aryl group preferably denoting a phenyl or benzyl group;

these compounds possibly being (poly)oxyalkylenated, notably (poly)oxyethylenated, and then preferably including from 1 to 50 ethylene oxide units and better still from 1 to 10 ethylene oxide units.

Preferentially, the sulfate anionic surfactants are chosen, alone or as a mixture, from:
alkyl sulfates, notably C₁₀-C₂₄ or even C₁₂-C₂₂ alkyl sulfates;
alkyl ether sulfates, notably C₁₀-C₂₄ or even C₁₂-C₂₂ alkyl ether sulfates, preferably comprising from 1 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Advantageously, the anionic surfactant(s) are chosen from sulfate anionic surfactants.

More preferentially, the anionic surfactant(s) are chosen from alkyl sulfates, alkyl ether sulfates, alkylamido ether sulfates, alkylaryl polyether sulfates, monoglyceride sulfates; mixtures thereof and the salts of these compounds;

the alkyl groups of these compounds including from 6 to 30 carbon atoms, notably from 8 to 28, even better still from 10 to 24 or even from 12 to 22 carbon atoms; the aryl group preferably denoting a phenyl or benzyl group;

these compounds possibly being (poly)oxyalkylenated, notably (poly)oxyethylenated, and then preferably including from 1 to 50 ethylene oxide units and better still from 1 to 10 ethylene oxide units.

Even more preferentially, the anionic surfactant(s) are chosen from:
C₆-C₃₀, better still C₅-C₂₄, even better still C₁₀-C₂₄ or even C₁₂-C₂₂ alkyl sulfates,
C₆-C₂₄, better still C₁₀-C₂₄ or even C₁₂-C₂₂ alkyl ether sulfates, preferably comprising from 1 to 20 ethylene oxide units,
and mixtures thereof;

in particular in the form of alkali metal or alkaline-earth metal, ammonium or amino alcohol salts.

Even more preferentially, the anionic surfactant(s) are chosen from C₆-C₃₀, better still C₅-C₂₄, even better still C₁₀-C₂₄ or even C₁₂-C₂₂ alkyl sulfates.

Preferably, the anhydrous solid composition (A) comprises at least two anionic surfactants, preferably at least two sulfate anionic surfactants, more preferentially at least two C₆-C₃₀, better still C₅-C₂₄, even better still C₁₀-C₂₄ or even C₁₂-C₂₂ alkyl sulfates.

Preferably, the total content of anionic surfactant(s) present in the anhydrous solid composition (A) ranges from 55% to 90% by weight, preferentially from 56% to 85% by weight, more preferentially from 57% to 80% by weight, even more preferentially from 58% to 75% by weight, better still from 59% to 75% by weight and even better still from 60% to 75% by weight, relative to the total weight of the anhydrous solid composition (A).

Preferably, the total content of sulfate anionic surfactant(s) present in the anhydrous solid composition (A) ranges from 55% to 90% by weight, preferentially from 56% to 85% by weight, more preferentially from 57% to 80% by weight, even more preferentially from 58% to 75% by weight, better still from 59% to 75% by weight and even better still from 60% to 75% by weight, relative to the total weight of the anhydrous solid composition (A).

Preferably, the total content of C₆-C₃₀ alkyl sulfates present in the anhydrous solid composition (A) ranges from 55% to 90% by weight, preferentially from 56% to 85% by weight, more preferentially from 57% to 80% by weight, even more preferentially from 58% to 75% by weight, better still from 59% to 75% by weight and even better still from 60% to 75% by weight, relative to the total weight of the anhydrous solid composition (A).

Amphoteric or Zwitterionic Surfactants

The anhydrous solid composition (A) comprises one or more amphoteric or zwitterionic surfactants.

In particular, the amphoteric or zwitterionic surfactant(s), which are preferably non-silicone, used in the anhydrous solid composition (A) may notably be derivatives of optionally quaternized secondary or tertiary aliphatic amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₁-C₆)alkylbetaines and (C₈-C₂₀)alkylamido(C₁-C₆)alkylsulfobetaines, and mixtures thereof.

Among the optionally quaternized derivatives of secondary or tertiary aliphatic amines that may be used, as defined above, mention may also be made of the compounds having the respective structures (III) and (IV) below:

Rₐ—CONHCH₂CH₂—N⁺(R_{b})(R_{c})—CH₂COO⁻,M⁺,X⁻ (III)

in which formula (III):
Rₐ represents a C₁₀ to C₃₀ alkyl or alkenyl group derived from an acid RₐCOOH preferably present in hydrolysed coconut kernel oil; preferably, Rₐ represents a heptyl, nonyl or undecyl group;
R_{b} represents a β-hydroxyethyl group;
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

Rₐ′—CONHCH₂CH₂—N(B)(B′) (IV)

in which formula (IV):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH, CH₂CH₂—COOZ′, or a hydrogen atom;
Y′ represents the group —COOH, —COOZ′, —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ′ represents a C₁₀ to C₃₀ alkyl or alkenyl group of an acid R_{a′}—COOH which is preferably present in coconut kernel oil or in hydrolysed linseed oil, preferably R_{a′} an alkyl group, notably a C₁₇ group, and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (V):

Rₐ″—NHCH(Y″)—(CH₂)ₙCONH(CH₂)_{n′}—N(R_{d})(Rₑ) (V)

in which formula (V):
Y″ represents the group —COOH, —COOZ″, —CH₂—CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a′}—COOH which is preferably present in coconut kernel oil or in hydrolysed linseed oil; and
n and n′ denote, independently of each other, an integer ranging from 1 to 3.

Among the compounds of formula (V), mention may be made of the compound classified in the CTFA dictionary under the name sodium diethylaminopropyl cocoaspartamide and sold by the company Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoylbetaine (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof.

More preferentially, the amphoteric or zwitterionic surfactants are chosen from (C₈-C₂₀)alkylbetaines, (C₈-C₂₀)alkylamido(C₃-C₈)alkylbetaines, and mixtures thereof, even more preferentially from cocoylbetaine, cocamidopropylbetaine, and mixtures thereof.

Better still, the amphoteric or zwitterionic surfactants are chosen from (C₈-C₂₀)alkylamido(C₃-C₈)alkylbetaines and most particularly cocamidopropylbetaine.

Preferably, the total content of amphoteric or zwitterionic surfactant(s) present in the anhydrous solid composition (A) ranges from 5% to 40% by weight, more preferentially from 6% to 35% by weight, even more preferentially from 7% to 30% by weight and better still from 8% to 20% by weight relative to the total weight of the anhydrous solid composition (A).

Preferably, the total content of (C₈-C₂₀)alkylbetaines and (C₈-C₂₀)alkylamido(C₃-C₈)alkylbetaines present in the anhydrous solid composition (A) ranges from 5% to 40% by weight, more preferentially from 6% to 35% by weight, even more preferentially from 7% to 30% by weight and better still from 8% to 20% by weight relative to the total weight of the anhydrous solid composition (A).

Preferably, the total content of (C₈-C₂₀)alkylamido(C₃-C₈)alkylbetaine(s), such as cocamidopropylbetaine, present in the anhydrous solid composition (A) ranges from 5% to 40% by weight, more preferentially from 6% to 35% by weight, even more preferentially from 7% to 30% by weight and better still from 8% to 20% by weight relative to the total weight of the anhydrous solid composition (A).

Advantageously, the total content of surfactant(s) present in the anhydrous solid composition (A) is greater than or equal to 60% by weight, preferably ranges from 60% to 95% by weight, more preferentially from 65% to 90% by weight and even more preferentially from 70% to 85% by weight, relative to the total weight of the anhydrous solid composition (A).

Preferably, the total content of anionic surfactant(s) and amphoteric or zwitterionic surfactant(s) present in the anhydrous solid composition (A) is greater than or equal to 60% by weight, more preferentially ranges from 60% to 95% by weight, even more preferentially from 65% to 90% by weight and better still from 70% to 85% by weight, relative to the total weight of the anhydrous solid composition (A).

Preferably, the total content of sulfate anionic surfactant(s) and amphoteric or zwitterionic surfactant(s) present in the anhydrous solid composition (A) is greater than or equal to 60% by weight, more preferentially ranges from 60% to 95% by weight, even more preferentially from 65% to 90% by weight and better still from 70% to 85% by weight, relative to the total weight of the anhydrous solid composition (A).

Preferably, the total content of C₆-C₃₀ alkyl sulfates and (C₈-C₂₀)alkylbetaines and (C₈-C₂₀)alkylamido(C₃-C₈)alkylbetaines present in the anhydrous solid composition (A) is greater than or equal to 60% by weight, more preferentially ranges from 60% to 95% by weight, even more preferentially from 65% to 90% by weight and better still from 70% to 85% by weight, relative to the total weight of the anhydrous solid composition (A).

Anticaking Agents

Preferably, the anhydrous solid composition (A) also comprises one or more anticaking agents.

For the purposes of the invention, the term “anticaking agent” means a compound for reducing or even preventing the agglomeration of the solid particles and/or of the powders, for reducing the adherence, and/or for improving the flow of the solid particles and/or of the powders by reducing the friction and cohesion between same.

Preferably, the anticaking agent(s) are chosen from C₈-C₃₂ fatty acid salts, tricalcium phosphate, calcium silicate, magnesium silicate, magnesium carbonate, silicon dioxide, talc, silica, sodium stearyl fumarate, tetrasodium pyrophosphate, and mixtures thereof.

The fatty acid salts that may be used according to the present invention advantageously comprise from 10 to 20 carbon atoms and more preferentially from 12 to 18 carbon atoms.

They are more particularly salts of fatty acids chosen from caprylic acid, capric acid, lauric acid, myristic acid, palmitic acid, stearic acid, oleic acid, and mixtures thereof, such as cocoate salts.

The fatty acid salts may be chosen from salts of alkali metals, of alkaline-earth metals or of amines. The salt may be chosen from sodium, potassium, calcium, magnesium, ammonium, diethanolamine, triethanolamine and triisopropanolamine salts.

As examples of C₈-C₃₂ fatty acid salts, mention may be made of:
sodium or potassium caprate,
sodium or potassium caprylate,
sodium or potassium or magnesium or calcium or ammonium or triethanolamine laurate,
sodium or potassium or magnesium or calcium or diethanolamine or triethanolamine or triisopropanolamine myristate,
sodium or potassium or magnesium or triethanolamine palmitate,
sodium or potassium or magnesium or triethanolamine cocoate,
sodium or potassium or magnesium or calcium or ammonium or diethanolamine or triethanolamine stearate,
sodium or potassium or ammonium oleate,
sodium arachidate,
sodium or potassium or calcium behenate.

Advantageously, use is made of a salt of a monocarboxylic acid, which is preferably saturated, containing from 8 to 32 carbon atoms, more preferentially from 10 to 20 carbon atoms, such as those described previously.

Preferably, the salt(s) of fatty acids including from 8 to 32 carbon atoms are chosen from stearic acid salts; more preferentially from the alkali metal and alkaline-earth metal salts of stearic acid, and mixtures thereof.

For the purposes of the present invention, it is understood that said anticaking agents, and in particular salts of C₈-C₃₂ fatty acids, are different from the anionic surfactant(s) (i) present in the composition according to the invention.

More preferentially, the anticaking agent(s) are chosen from salts of C₈-C₃₂, better still C₁₀-C₂₀ and even better still C₁₂-C₁₈ fatty acids; even more preferentially from the alkali metal and alkaline-earth metal salts of stearic acid, and mixtures thereof, and better still magnesium stearate.

Preferably, when the anticaking agent(s) are present in the anhydrous solid composition (A), the total content of anticaking agent(s) ranges from 0.01% to 20% by weight, more preferentially from 0.1% to 15% by weight, even more preferentially from 0.5% to 10% by weight and better still from 1% to 8% by weight relative to the total weight of the anhydrous solid composition (A).

Preferably, when the salt(s) of C₈-C₃₂ fatty acids are present in the anhydrous solid composition (A), the total content of salt(s) of C₈-C₃₂ fatty acids ranges from 0.01% to 20% by weight, more preferentially from 0.1% to 15% by weight, even more preferentially from 0.5% to 10% by weight and better still from 1% to 8% by weight relative to the total weight of the anhydrous solid composition (A).

Preferably, when magnesium stearate is present in the anhydrous solid composition (A), the total content of magnesium stearate ranges from 0.01% to 20% by weight, more preferentially from 0.1% to 15% by weight, even more preferentially from 0.5% to 10% by weight and better still from 1% to 8% by weight relative to the total weight of the anhydrous solid composition (A).

Cationic Polymers

Preferably, the anhydrous solid composition (A) also comprises one or more cationic polymers.

For the purposes of the present invention, the term “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers are not silicone-based (they do not comprise any Si—O units).

The cationic polymers may be associative or non-associative.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:

(1) homopolymers or copolymers derived from acrylic or methacrylic esters or amides and including at least one of the units having the following formulae:

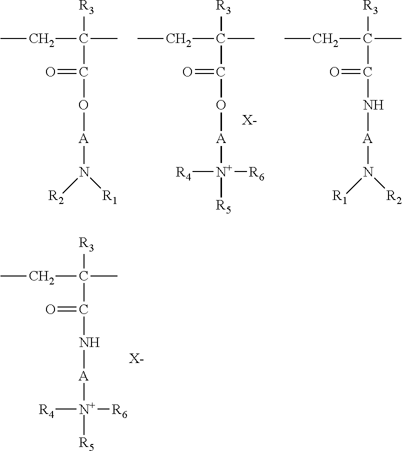

in which formulae:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, and preferably an alkyl group containing from 1 to 6 carbon atoms;
R₁ and R₂, which may be identical or different, represent a hydrogen atom or an alkyl group containing from 1 to 6 carbon atoms, preferably methyl or ethyl; and
X denotes an anion derived from a mineral or organic acid, such as a methosulfate anion or a halide such as chloride or bromide.

The copolymers of the family (1) may also contain one or more units derived from comonomers which may be chosen from the family of acrylamides, methacrylamides, diacetone acrylamides, acrylamides and methacrylamides substituted on the nitrogen with lower alkyls (C₁-C₄), acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers such as the product sold under the name Gafquat HS 100 by the company ISP;
polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.

(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group, for instance Polyquaternium-10.

Cationic cellulose copolymers or cellulose derivatives grafted with a water-soluble quaternary ammonium monomer are notably described in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt, for instance Polyquaternium-4. The commercial products corresponding to this definition are more particularly the products sold under the names Celquat L 200 and Celquat H 100 by the company National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (Ib):

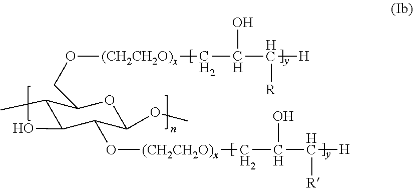

in which:
R represents an ammonium group RaRbRcN⁺, Q⁻ in which Ra, Rb and Rc, which may be identical or different, represent a hydrogen atom or a linear or branched C₁ to C₃₀ alkyl, preferably an alkyl, and Q⁻ represents an anionic counterion such as a halide, for instance a chloride or bromide;
R′ represents an ammonium group R′aR′bR′cN⁺—, Q′⁻ in which R′a, R′b and R′c, which may be identical or different, represent a hydrogen atom or a linear or branched C₁ to C₃₀ alkyl, preferably an alkyl, and Q′⁻ represents an anionic counterion such as a halide, for instance a chloride or bromide;

it being understood that at least one of the radicals Ra, Rb, Rc, R′a, R′b, R′c represents a linear or branched C₈ to C₃₀ alkyl;
n, x and y, which may be identical or different, represent an integer between 1 and 10 000.

Preferably, in formula (Ib), at least one of the radicals Ra, Rb, Rc, R′a, R′b, R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Ra, Rb, Rc, R′a, R′b, R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Better still, R may be a group chosen from —N⁺(CH₃)₃, Q′⁻ and

—N⁺(C₁₂H₂₅)(CH₃)₂, Q′⁻, preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 et SL-5 from the company Amerchol/Dow Chemical.

More particularly, the polymers of formula (Ib) are, for example, those whose viscosity is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17, Jaguar C162 or Jaguar Excel by the company Rhodia. Such compounds have the INCI name guar hydroxypropyltrimonium chloride or hydroxypropyl guar hydroxypropyltrimonium chloride.

(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.

(4) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they include one or more tertiary amine functions, they can be quaternized;

(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.

(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or else under the name PD 170 or Delsette 101 by the company Hercules in the case of the adipic acid/epoxypropyl/diethylenetriamine copolymer.

(7) cyclopolymers of alkyldiallylamine or of dialkyldiallylammonium, such as homopolymers or copolymers including, as main constituent of the chain, units corresponding to formula (VI) or (VII):

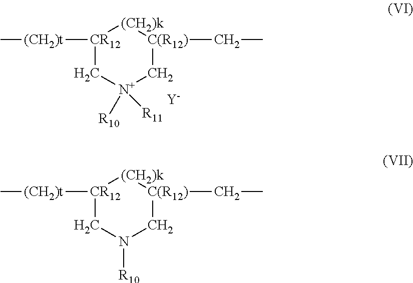

in which formulae (VI) and (VII):
k and t are equal to 0 or 1, the sum k+t being equal to 1;
R₁₂ denotes a hydrogen atom or a methyl radical;
R₁₀ and R₁₁, independently of each other, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of each other, preferably denote an alkyl group containing from 1 to 4 carbon atoms; and
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold, for example, under the name Merquat 100 by the company Nalco (and homologues thereof of low weight-average molar masses) and the copolymers of diallyldimethylammonium salts (for example chloride) and of acrylamide, notably sold under the names Merquat 550 and Merquat 7SPR.

(8) quaternary diammoniumpolymers comprising repeating units of formula:

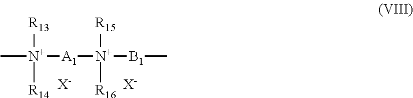

in which formula (VIII):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;

it being understood that A₁, R₁₃ and R₁₅ can form, with the two nitrogen atoms to which they are attached, a piperazine ring;

in addition, if A₁ denotes a linear or branched, saturated or unsaturated alkylene or hydroxyalkylene radical, B₁ can also denote a group (CH₂)ₙCO-D-OC—(CH₂)ₙ— in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— et —[CH₂—CH(CH₃)—O]_{y}—CH₂—CH(CH₃)— in which x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or else the divalent radical-CH₂—CH₂—S—S—CH₂—CH₂—; or
d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers consisting of repeating units corresponding to the formula:

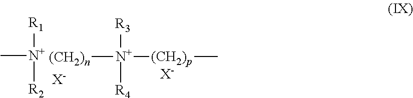

in which formula (IX) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (IX) that is particularly preferred is the one for which R₁, R₂, R₃ and R₄ represent a methyl radical and n=3, p=6 and X═Cl, which is known as Hexadimethrine chloride according to the INCI (CTFA) nomenclature.

(9) polyquaternary ammonium polymers comprising units of formula (X):

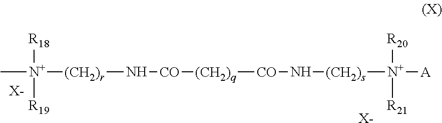

in which formula (X):
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH, radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.

(10) quaternary polymers of vinylpyrrolidone and of vinylimidazole, for instance the products sold under the names Luviquat® FC 905, FC 550 and FC 370 by the company BASF.

(11) polyamines such as Polyquart® H sold by Cognis, which is referenced under the name Polyethylene Glycol (15) Tallow Polyamine in the CTFA dictionary.

(12) polymers including in their structure:
(a) one or more units corresponding to formula (A) below:

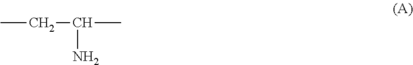

(b) optionally one or more units corresponding to formula (B) below:

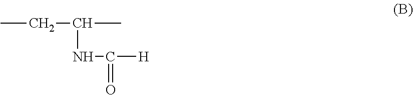

In other words, these polymers may be notably chosen from homopolymers or copolymers including one or more units derived from vinylamine and optionally one or more units derived from vinylformamide.

(13) and mixtures thereof.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may preferably range from 1000 à 3.000.000 g/mol, more preferentially from 10 000 à 1.000.000 and more particularly from 100 000 à 500.000 g/mol.

The cationic charge density of these polymers may preferably range from 2 meq/g to 20 meq/g, more preferentially from 2.5 to 15 meq/g and more particularly from 3.5 to 10 meq/g.

The polymers including units of formula (A) and optionally units of formula (B) are notably sold under the name Lupamin by the company BASF, for instance, in a non-limiting manner, the products sold under the names Lupamin 9095, Lupamin 5095, Lupamin 1095, Lupamin 9030 (or Luviquat 9030) and Lupamin 9010.

Preferably, the cationic polymer(s) are chosen from cationic polysaccharides.

More preferentially, the cationic polymer(s) are chosen from cellulose ether derivatives including quaternary ammonium groups, cationic cellulose copolymers, cellulose derivatives grafted with a water-soluble quaternary ammonium monomer and cationic galactomannan gums, and mixtures thereof.

Even more preferentially, the cationic polymer(s) are chosen from cationic galactomannan gums, quaternary ammonium polymers of hydroxyethylcellulose which have reacted with an epoxide substituted with a trimethylammonium group, and mixtures thereof.

Most particularly preferably, the cationic polymer(s) are chosen from cationic guar gums, Polyquaternium-10, and mixtures thereof.

Preferably, when the cationic polymer(s) are present in the anhydrous solid composition (A), the total content of cationic polymer(s) ranges from 0.1% to 20% by weight, more preferentially from 0.5% to 15% by weight, even more preferentially from 1% to 10% by weight and better still from 2% to 5% by weight relative to the total weight of the anhydrous solid composition (A).

Preferably, when the cationic polysaccharide(s) are present in the anhydrous solid composition (A), the total content of cationic polysaccharide(s) ranges from 0.1% to 20% by weight, more preferentially from 0.5% to 15% by weight, even more preferentially from 1% to 10% by weight and better still from 2% to 5% by weight relative to the total weight of the anhydrous solid composition (A).

Preferably, when the cationic galactomannan gum(s) and/or the quaternary ammonium polymer(s) of hydroxyethylcellulose that have reacted with an epoxide substituted with a trimethylammonium group are present in the anhydrous solid composition (A), the total content of cationic galactomannan gum(s) and of quaternary ammonium polymer(s) of hydroxyethylcellulose that have reacted with an epoxide substituted with a trimethylammonium group ranges from 0.1% to 20% by weight, more preferentially from 0.5% to 15% by weight, even more preferentially from 1% to 10% by weight and better still from 2% to 5% by weight relative to the total weight of the anhydrous solid composition (A).

Nonionic Surfactants

The anhydrous solid composition (A) may also optionally comprise one or more nonionic surfactants.

The nonionic surfactants that may be used according to the invention may be chosen from:
alcohols, α-diols and (C₁-C₂₀)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; and/or these compounds comprising at least one fatty chain including from 8 to 40 carbon atoms and notably from 10 to 20 carbon atoms; in particular, oxyethylenated and/or oxypropylenated alcohols comprising at least one saturated or unsaturated, linear or branched C₈ to C₄₀ alkyl chain, comprising from 1 to 100 mol of ethylene oxide and/or of propylene oxide, preferably from 2 to 50 and more particularly from 2 to 40 mol of ethylene oxide and/or of propylene oxide;
condensates of ethylene oxide and propylene oxide with fatty alcohols;
polyethoxylated fatty amides preferably containing from 2 to 30 ethylene oxide units, polyglycerolated fatty amides including on average from 1 to 5 and in particular from 1.5 to 4 glycerol groups;
ethoxylated fatty acid esters of sorbitan, preferably containing from 2 to 40 ethylene oxide units;
fatty acid esters of sucrose;
polyoxyalkylenated, preferably polyoxyethylenated, fatty acid esters containing from 2 to 150 mol of ethylene oxide, including oxyethylenated plant oils;
N—(C₆-C₂₄ alkyl)glucamine derivatives;
amine oxides such as (C₁₀-C₁₄ alkyl)amine oxides or N—(C₁₀-C₁₄ acyl)aminopropylmorpholine oxides;
and mixtures thereof.

Mention may also be made of nonionic surfactants of alkyl(poly)glycoside type, represented notably by the following general formula:

R₁O—(R₂O)ₜ-(G)ᵥ

in which:
R₁ represents a linear or branched alkyl or alkenyl radical including 6 to 24 carbon atoms and notably 8 to 18 carbon atoms, or an alkylphenyl radical of which the linear or branched alkyl radical includes 6 to 24 carbon atoms and notably 8 to 18 carbon atoms,
R₂ represents an alkylene radical including 2 to 4 carbon atoms,
G represents a sugar unit including 5 to 6 carbon atoms,
t denotes a value ranging from 0 to 10 and preferably from 0 to 4,
v denotes a value ranging from 1 to 15 and preferably from 1 to 4.

Preferably, the alkyl(poly)glycoside surfactants are compounds of the formula described above in which:
R₁ denotes a linear or branched, saturated or unsaturated alkyl radical including from 8 to 18 carbon atoms,
R₂ represents an alkylene radical including 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. 1,4 C₈/C₁₆-alkyl(poly)glucosides, and notably decyl glucosides and caprylyl/capryl glucosides, are most particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or the products sold by the company Chem Y under the name AG10 LK.

Preferably, use is made of 1,4 C₈/C₁₆-alkyl(poly)glycosides, notably as an aqueous 53% solution, such as those sold by Cognis under the reference Plantacare® 818 UP.

Preferentially, the nonionic surfactant(s) are chosen from oxyalkylenated fatty alcohols including at least one saturated or unsaturated, linear or branched C₈ to C₄₀ alkyl chain and comprising a number of ethylene oxide and/or propylene oxide groups ranging from 1 to 100; more preferentially chosen from oxyethylenated and oxypropylenated fatty alcohols including at least one saturated or unsaturated, linear or branched C₈ to C₂₀ and better still C₁₀ to C₁₈ alkyl chain and comprising a number of ethylene oxide and propylene oxide groups ranging from 2 to 50, better still from 2 to 40, such as PPG-5-Ceteth-20.

Preferably, when the nonionic surfactant(s) are present in the anhydrous solid composition (A), the total content of nonionic surfactant(s) ranges from 0.1% to 15% by weight, more preferentially from 0.5% to 10% by weight, even more preferentially from 1% to 5% by weight relative to the total weight of the anhydrous solid composition (A).

Preferably, when the oxyalkylenated fatty alcohol(s) are present in the anhydrous solid composition (A), the total content of oxyalkylenated fatty alcohol(s) ranges from 0.1% to 15% by weight, more preferentially from 0.5% to 10% by weight, even more preferentially from 1% to 5% by weight relative to the total weight of the anhydrous solid composition (A).

The anhydrous solid composition may also optionally comprise one or more silicones, preferably chosen from amino silicones; notably in a content ranging from 0.01% to 10% by weight, more preferentially from 0.1% to 7% by weight and even more preferentially from 0.5% to 5% by weight, relative to the total weight of the anhydrous solid composition.

The term “silicone” means any organosilicon polymer or oligomer of linear or cyclic and branched or crosslinked structure, of variable molecular weight, obtained by polymerization and/or polycondensation of suitably functionalized silanes and consisting essentially of a repetition of main units in which the silicon atoms are connected to each other via oxygen atoms (siloxane bond —Si—O—Si—), optionally substituted hydrocarbon-based radicals being connected directly to said silicon atoms via a carbon atom; and more particularly dialkylsiloxane polymers, amino silicones and dimethiconols.

The term “amino silicone” denotes any silicone including at least one primary, secondary or tertiary amine or a quaternary ammonium group.

The anhydrous solid composition (A) may also contain additives usually used in cosmetics, for instance preserving agents, fragrances and colorants.

These additives, preferably in the form of powders, may be present in composition (A) in an amount ranging from 0 to 20% by weight, relative to the total weight of the composition.

A person skilled in the art will take care to select these optional additives and the amounts thereof such that they do not harm the properties of the anhydrous solid composition (A).

According to a preferred embodiment of the invention, the anhydrous solid composition (A) comprises:
(i) one or more sulfate anionic surfactants,
(ii) one or more amphoteric or zwitterionic surfactants chosen from (C₈-C₂₀)alkylbetaines, (C₈-C₂₀)alkylamido(C₃-C₈)alkylbetaines, and mixtures thereof, and
(iii) one or more salts of a C₁₀-C₂₀ fatty acid; and
the total content of surfactants being greater than or equal to 60% by weight relative to the total weight of the anhydrous solid composition.

According to a particularly preferred embodiment of the invention, the anhydrous solid composition (A) comprises:
(i) one or more sulfate anionic surfactants,
(ii) one or more amphoteric or zwitterionic surfactants chosen from (C₈-C₂₀)alkylbetaines, (C₈-C₂₀)alkylamido(C₃-C₈)alkylbetaines, and mixtures thereof, (iii) one or more salts of a C₁₀-C₂₀ and better still C₁₂-C₁₈ fatty acid, even more preferentially magnesium stearate,
(iv) optionally one or more cationic polymers, preferably chosen from cationic polysaccharides, more preferentially chosen from cationic guar gums, Polyquaternium-10, and mixtures thereof, and
(v) optionally one or more nonionic surfactants, preferably one or more oxyalkylenated C₅-C₄₀ fatty alcohols;
the total content of surfactants being greater than or equal to 60% by weight, preferably ranging from 60% to 95% by weight, better still from 65% to 90% by weight and even better still from 70% to 85% by weight, relative to the total weight of the anhydrous solid composition.

Composition (B)

The preparation process according to the invention comprises at least one step of mixing an anhydrous solid composition (A) with an aqueous composition (B).

Composition (B) used in the process according to the invention comprises water.

Preferably, the total content of water in composition (B) ranges from 80% to 100% by weight, more preferentially from 85% to 100% by weight and even more preferentially from 90% to 100% by weight, relative to the total weight of composition (B).

More preferentially, composition (B) comprises 100% water.

Composition (B) may also contain additives usually used in cosmetics, for instance preserving agents, fragrances and/or colorants.

These additives may be present in composition (B) in an amount ranging from 0 to 20% by weight, relative to the total weight of the composition.

A person skilled in the art will take care to select these optional additives and the amounts thereof so that they do not harm the properties of composition (B).

The weight ratio of the total content of anhydrous solid composition (A), on the one hand, to the total content of composition (B), on the other hand, ranges from 0.01 to 0.5, preferably from 0.05 to 0.4 and more preferentially from 0.1 to 0.3.

Composition (C) corresponds to the aqueous composition obtained by mixing composition (A) with composition (B) described previously.

Preferably, the duration of the agitation in step (ii) of the preparation process according to the invention ranges from 1 to 120 seconds; more preferentially from 2 to 60 seconds; even more preferentially from 3 to 30 seconds; better still from 5 to 20 seconds.

The agitation of the aqueous composition (C) may be performed by hand, for example by shaking and/or inverting the container containing composition (C) one or more times.

Advantageously, once step (ii) has been performed, the anhydrous solid composition (A) may be totally dissolved.

In step (iii) of the preparation process according to the invention, composition (C) is left to stand for at least 20 minutes at room temperature (25° C.) and atmospheric pressure, more preferentially at least 25 minutes, even more preferentially 30 minutes, better still 60 minutes, even better still 90 minutes, better still 2 hours, better still 4 hours, better still 6 hours, better still 8 hours, better still 10 hours, or even 12 hours, or even 18 hours, and most particularly preferably at least 24 hours, before it is used on the keratin materials.

The standing time notably makes it possible to debubble the composition and may thus lead to a composition that is more transparent or translucent when it is left to stand for longer.

Preferably, the process according to the invention is a process for preparing a composition for washing keratin materials, more preferentially for washing the hair.

Ready-to-Use Composition

The aqueous composition obtained via the preparation process according to the invention is ready to use.

The aqueous composition obtained via the preparation process according to the invention is preferably a shampoo.

Preferably, the pH of the ready-to-use aqueous composition obtained via the preparation process according to the invention is between 3 and 8, more preferentially between 3.5 and 7 and even more preferentially between 4.5 and 5.5.

More preferentially, the process according to the invention is a process for preparing a transparent composition for washing keratin materials.

The aqueous composition obtained via the preparation process according to the invention is preferably transparent

It has been noted that the transparency of the ready-to-use composition is particularly aesthetic and sought by users, in particular when the aqueous composition obtained is packaged in a transparent container.

The term “transparent composition” means a composition through which it is possible to see distinctly with the naked eye.

The transparency of the ready-to-use composition may be characterized by measuring its transmittance.

In the context of the present invention, the transmittance measurements are performed at 25° C. and at atmospheric pressure, with a Cary Type 100 Scan UV-visible spectrophotometer.

Preferably, the transmittance of the ready-to-use composition, measured at room temperature (25° C.) and atmospheric pressure, is greater than or equal to 80%, preferentially greater than or equal to 85%, even more preferentially greater than or equal to 90%, better still greater than or equal to 92%; and in particular ranging from 80% to 100%, or even from 85% to 100%, notably from 90% to 100%, or even from 92% to 100%.

The invention also relates to the process for treating keratin materials, comprising the application to said keratin materials of the composition obtained via the preparation process as described previously.

Preferably, the treatment process is a process for washing keratin materials, and optionally also for conditioning keratin materials.

The examples that follow serve to illustrate the invention without, however, being limiting in nature.

### EXAMPLES

### Example 1

The anhydrous solid compositions A to F according to the invention are prepared from the ingredients indicated in the tables below, the amounts of which are expressed as weight percentages of active material (AM).

TABLE 1

| | Composition A | Composition B |
|---|---|---|
| Sodium lauryl sulfate | 42.1 | 59.2 |
| Sodium cocoyl sulfate | 20.8 | — |
| Cocamidopropylbetaine | 16.2 | 20.9 |
| Hydroxypropyl guar | 3.3 | — |
| hydroxypropyltrimonium | | |
| chloride | | |
| Polyquaternium-10 | — | 3.1 |
| PPG-5-Ceteth-20 | 2.5 | 2.6 |
| Magnesium stearate | 3 | 1 |
| Preserving agents | 5.9 | 4.8 |
| Ethyl lactate | — | 1 |
| Sodium chloride | 2.9 | 3.7 |
| Fragrance | 3.3 | 3.7 |

TABLE 2

| | Composition C | Composition D |
|---|---|---|
| Sodium lauryl sulfate | 70.5 | 68.9 |
| Cocamidopropylbetaine | 9.1 | 8.9 |
| Hydroxypropyl guar | 3.1 | 4.8 |
| hydroxypropyltrimonium | | |
| chloride | | |
| Hydroxypropyltrimonium guar | 1.8 | — |
| chloride | | |
| Amodimethicone | — | 3 |
| Magnesium stearate | 3 | 3 |
| Preserving agents | 7.2 | 6.3 |
| Sodium chloride | 1.6 | 1.6 |
| Fragrance | 3.7 | 3.5 |

TABLE 3

| | Composition E |
|---|---|
| Sodium lauryl sulfate | 64.1 |
| Cocamidopropylbetaine | 16.5 |
| Hydroxypropyl guar | 3.3 |
| hydroxypropyltrimonium | |
| chloride | |
| Mica | 0.7 |
| Tricalcium phosphate | 3 |
| Preserving agents | 6.1 |
| Sodium chloride | 3 |
| Fragrance | 3.3 |

TABLE 4

| | Composition F |
|---|---|
| Sodium lauryl sulfate | 65.8 |
| Cocamidopropylbetaine | 14.3 |
| Hydroxypropyl guar | 1.7 |
| hydroxypropyltrimonium | |
| chloride | |
| Polyquaternium-10 | 1.7 |
| Magnesium stearate | 5 |
| Preserving agents | 6.9 |
| Sodium chloride | 1.2 |
| Fragrance | 3.4 |

Protocol:

Each anhydrous solid composition (A to F) according to the invention was poured into a different transparent glass bottle comprising water, in the respective amounts indicated in Table 5 below.

TABLE 5

| | Solid compositions | | | | | |
|---|---|---|---|---|---|---|
| | A | B | C | D | E | F |
| Amount of | 45.7 | 48.6 | 40.8 | 41.7 | 44.9 | 43.7 |
| anhydrous solid | | | | | | |
| composition | | | | | | |
| added (in g) | | | | | | |
| Amount of | 204.3 | 201.4 | 209.2 | 208.3 | 205.1 | 206.3 |
| water added | | | | | | |
| (in g) | | | | | | |
| Weight ratio of | 0.22 | 0.24 | 0.20 | 0.20 | 0.22 | 0.21 |
| the amount of | | | | | | |
| anhydrous solid | | | | | | |
| composition | | | | | | |
| to the amount | | | | | | |
| of water | | | | | | |

The six closed bottles were then shaken and inverted several times by hand to obtain, respectively, the aqueous compositions M_{A} to M_{F}.

The aqueous compositions M_{A} to M_{F} were then left to stand for 24 hours at room temperature (25° C.) and at atmospheric pressure.

After leaving to stand for 24 hours, it is observed that the aqueous compositions M_{A} to M_{D} are transparent, and that the aqueous compositions M_{E} to M_{F} are opaque.

It was notably observed that the aqueous composition M_{A} has a transmittance of 97%.

After leaving to standard for 24 hours, the aqueous compositions M_{A} to M_{F} were applied to locks of hair (2 g of composition/g of lock of hair). The locks were then left in the open air for a leave-on time of 5 minutes, and were finally rinsed with clean water.

It was observed that the aqueous compositions M_{A} to M_{F} have good washing power and give the hair good cosmetic properties. In particular, these aqueous compositions M_{A} to M_{F} afford suppleness, a soft feel, sheen and ease of disentangling to the hair.

### Example 2

The anhydrous solid composition G according to the invention and the comparative anhydrous solid composition H are prepared from the ingredients indicated in the tables below, the amounts of which are expressed as weight percentages of active material (AM).

TABLE 6

| | Composition G | Composition H |
|---|---|---|
| | (Invention) | (Comparative) |
| Sodium lauryl sulfate | 60 | 75.2 |
| Cocamidopropylbetaine | 15.2 | — |
| Hydroxypropyl guar | 2.9 | 2.9 |
| hydroxypropyltrimonium chloride | | |
| Sodium benzoate | 3.6 | 3.6 |
| Citric acid | 4.6 | 4.6 |
| Magnesium stearate | 5 | 5 |
| Sodium chloride | 4.4 | 4.4 |
| Fragrance | 4.3 | 4.3 |

Protocol:

Each anhydrous solid composition G and H was poured into a different transparent glass bottle comprising water, in the following amounts: 13.9 g of composition+86.1 g of water.

The two closed bottles were then shaken and inverted several times by hand to obtain, respectively, the aqueous compositions M_{G} (invention) and M_{H} (comparative).

The aqueous compositions M_{G} and M_{H} were then left to stand for 12 hours at room temperature (25° C.) and at atmospheric pressure.

Evaluation and Results:

1/The Ford cup viscosity (diameter 8 mm) of each composition is measured after preparation. This method consists in measuring, at a determined temperature, the flow time of 90 g of composition through an orifice of determined diameter. The result is expressed in seconds.

| M_{G} | M_{H} |
|---|---|
| (invention) | (comparative) |
| 70 s | 4 s |

It is observed that the comparative aqueous composition M_{H}, obtained from the comparative anhydrous solid composition H, exhibits a very low viscosity, incompatible with good properties of use (a very low viscosity causes runs between the fingers, difficulties of homogeneous distribution on the hair, etc. . . . ), unlike the aqueous composition M_{G} according to the invention.

2/The aqueous compositions M_{G} and M_{H} were applied to locks of moderately sensitized hair (alkaline solubility=20%, SA20) at a rate of 0.4 g of composition per gram of hair. The aqueous compositions M_{G} and M_{H} are left on the hair for 30 s, then rinsed with water.

The cosmetic performances (smooth to the touch, softness and flexibility) were evaluated on wet hair by an expert, during a blind test, assigning a:

“+” to the lock of hair with the best results

“−” to the lock of hair with the least good results.

The evaluation of flexibility is tactile. We take the locks with both hands and try to bend them. We test the ability of the hair to bend easily, its malleability.

To evaluate the smoothness as well as softness, you grab the lock between your thumb and forefinger and slide your fingers along the lock from the top to the tips.

We evaluate whether the hair is soft; for the smooth feel, we evaluate whether the hair has any roughness, whether it catches the fingers, whether the feel is uniform from root to tip.

| | M_{G} | M_{H} |
|---|---|---|
| | (invention) | (comparative) |
| Softness | + | − |
| Smoothness | + | − |
| Flexibility | + | − |

The aqueous composition M_{G} according to the invention exhibits improved performance in terms of softness, smoothness and flexibility compared to the comparative aqueous composition M_{H}.

1. Process for preparing a composition for washing keratin materials, comprising:
(i) at least one step of mixing:
a. an anhydrous solid composition (A) comprising:
one or more anionic surfactants, and
one or more amphoteric or zwitterionic surfactants,
b. with a composition (B) comprising water,
the weight ratio of the total content of composition (A) to the total content of composition (B) ranging from 0.01 to 0.5; and then
(ii) at least one step of agitating the aqueous composition (C) resulting from said mixing; and then
(iii) at least one step consisting in leaving the aqueous composition (C) to stand for at least 20 minutes before using it on the keratin materials.

2. Process according to claim 1, characterized in that the anionic surfactant(s) are chosen from sulfate anionic surfactants;
preferably from alkyl sulfates, alkyl ether sulfates, alkylamido ether sulfates, alkylaryl polyether sulfates, monoglyceride sulfates; mixtures thereof and the salts of these compounds;
more preferentially from C₆-C₃₀, better still C₅-C₂₄, even better still C₁₀-C₂₄ or even C₁₂-C₂₂ alkyl sulfates, C₆-C₂₄, better still C₁₀-C₂₄, or even C₁₂-C₂₂ alkyl ether sulfates, preferably comprising from 1 to 20 ethylene oxide units, and mixtures thereof,
even more preferentially from C₆-C₃₀, better still C₈-C₂₄, even better still C₁₀-C₂₄ or even C₁₂-C₂₂ alkyl sulfates.

3. Process according to claim 1, characterized in that the total content of anionic surfactant(s) ranges from 55% to 90% by weight, preferably from 56% to 85% by weight, more preferentially from 57% to 80% by weight, even more preferentially from 58% to 75% by weight, better still from 59% to 75% by weight and even better still from 60% to 75% by weight, relative to the total weight of the anhydrous solid composition (A).

4. Process according to claim 1, characterized in that the amphoteric or zwitterionic surfactant(s) are chosen from (C8-C20)alkylbetaines, (C8-C20)alkylamido(C3-C8)alkylbetaines, (C8-C20)alkylamphoacetates, (C8-C20)alkylamphodiacetates and mixtures thereof; preferably from (C8-C20)alkylbetaines, (C8-C20)alkylamido(C3-C8)alkylbetaines and mixtures thereof.

5. Process according to claim 1, characterized in that the total content of amphoteric or zwitterionic surfactant(s) ranges from 5% to 40% by weight, preferably from 6% to 35% by weight, more preferentially from 7% to 30% by weight and better still from 8% to 20% by weight relative to the total weight of the anhydrous solid composition (A).

6. Process according to claim 1, characterized in that the total content of surfactant(s) is greater than or equal to 60% by weight, preferably ranges from 60% to 95% by weight, more preferentially from 65% to 90% by weight and even more preferentially from 70% to 85% by weight, relative to the total weight of the anhydrous solid composition (A).

7. Process according to claim 1, characterized in that the anhydrous solid composition (A) also comprises one or more anticaking agents, preferably chosen from salts of C₈-C₃₂ fatty acids, tricalcium phosphate, calcium silicate, magnesium silicate, magnesium carbonate, silicon dioxide, talc, silica, sodium stearyl fumarate, tetrasodium pyrophosphate, and mixtures thereof; preferably from salts of a C₈-C₃₂, better still C₁₀-C₂₀ and even better still C₁₂-C₁₈ fatty acid; more preferentially from the alkali metal and alkaline-earth metal salts of stearic acid, and mixtures thereof; and even more preferentially, the anticaking agent is magnesium stearate.

8. Process according to claim 1, characterized in that the anhydrous solid composition (A) also comprises one or more cationic polymers, preferably chosen from:
(1) homopolymers or copolymers derived from acrylic or methacrylic esters or amides and including at least one of the units having the following formulae: 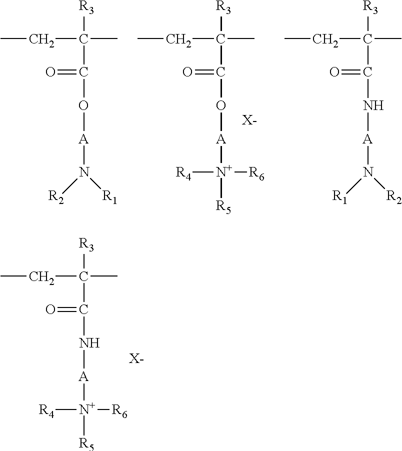
in which formulae:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, and preferably an alkyl group containing from 1 to 6 carbon atoms;
R₁ and R₂, which may be identical or different, represent a hydrogen atom or an alkyl group containing from 1 to 6 carbon atoms, preferably methyl or ethyl; and
X denotes an anion derived from a mineral or organic acid, such as a methosulfate anion or a halide such as chloride or bromide;
(2) cationic polysaccharides, such as cellulose ether derivatives including quaternary ammonium groups, cationic cellulose copolymers, cellulose derivatives grafted with a water-soluble quaternary ammonium monomer and cationic galactomannan gums;
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(4) water-soluble polyamino amides prepared in particular by polycondensation of an acidic compound with a polyamine;
(5) polyamino amide derivatives resulting from the condensation of polyalkylene polyamines with polycarboxylic acids followed by alkylation with difunctional agents;
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms;
(7) alkyldiallylamine or dialkyldiallylammonium cyclopolymers;
(8) quaternary diammonium polymers comprising repeating units of formula (VIII): 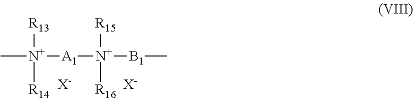
in which formula (VIII):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ may also denote a group (CH₂)ₙ—CO-D-OC—(CH₂)ₙ— in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— et —[CH₂—CH(CH₃)—O]_{y}—CH₂—CH(CH₃)— in which x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
d) a ureylene group of formula: —NH—CO—NH—,
(9) polyquaternary ammonium polymers comprising units of formula (X): 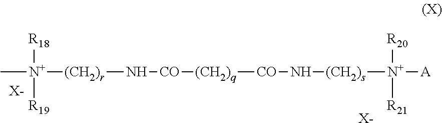
in which formula (X):
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH, radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—;
(10) quaternary polymers of vinylpyrrolidone and of vinylimidazole;
(11) polyamines; and
(12) polymers including in their structure:
(a) one or more units corresponding to formula (A) below: 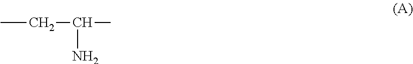
(b) optionally one or more units corresponding to formula (B) below: 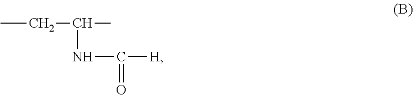
(13) and mixtures thereof;
more preferentially, the cationic polymer(s) are chosen from cationic polysaccharides.

9. Process according to claim 1, characterized in that the anhydrous solid composition (A) also comprises at least one nonionic surfactant; preferably chosen from oxyalkylenated fatty alcohols including at least one saturated or unsaturated, linear or branched C₈ to C₄₀ alkyl chain and comprising a number of ethylene oxide and/or propylene oxide groups ranging from 1 to 100; more preferentially chosen from oxyethylenated and oxypropylenated fatty alcohols including at least one saturated or unsaturated, linear or branched C₈ to C₂₀ and better still C₁₀ to C₁₈ alkyl chain and comprising a number of ethylene oxide and propylene oxide groups ranging from 2 to 50, better still from 2 to 40.

10. Process according to claim 1, characterized in that the anhydrous solid composition (A) is in powder form.

11. Process according to claim 1, characterized in that the weight ratio of the total content of anhydrous solid composition (A) to the total content of composition (B) ranges from 0.05 to 0.4, preferably from 0.1 to 0.3.

12. Process according to claim 1, characterized in that the duration of the agitation in step (ii) ranges from 1 to 120 seconds; preferably from 2 to 60 seconds; more preferentially from 3 to 30 seconds; better still from 5 to 20 seconds.

13. Process according to claim 1, characterized in that, in step (iii), composition (C) is left to stand for at least 30 minutes before it is used on the keratin materials; preferably at least 60 minutes, better still 90 minutes, even better still 2 hours, or even 4 hours, and most particularly preferably at least 24 hours before it is used on the keratin materials.

14. Process according to claim 1, for the preparation of a transparent composition for washing keratin materials, preferably the hair.

15. Process for treating keratin materials, comprising the application to said keratin materials of the composition obtained via the preparation process according to claim 1.
